(19) United States
(12) Patent Application Publication
(10) Pub. No.: US 2019/0038587 A1
(43) Pub. Date: Feb. 7, 2019

(54) DNA Methylation Landscapes of Post-Traumatic Stress Disorder (PTSD) Susceptibility and Resilience and Novel Therapeutics of PTSD Derived Thereof

(71) Applicants: Moshe Szyf, Quebec (CA); Gal Yadid, Shoham (IL); Gal Warhaftig, Haifa (IL)

(72) Inventors: Moshe Szyf, Quebec (CA); Gal Yadid, Shoham (IL); Gal Warhaftig, Haifa (IL)

(21) Appl. No.: 16/084,569

(22) PCT Filed: Feb. 13, 2017
(86) PCT No.: PCT/IL17/50180

§ 371 Date: Sep. 13, 2018

Related U.S. Application Data
(60) Provisional application No. 62/315,513, filed on Mar. 30, 2016.

(51) Int. Cl. A61K 31/203; A61K 31/335; A61K 31/4184; A61K 31/4178; A61K 31/385; A61K 31/557; A61P 25/18; G06F 19/28; C12Q 1/6883

(52) U.S. Cl. CPC A61K 31/203; A61K 31/335; A61K 31/4184; A61K 31/4178; A61K 31/7076; A61K 31/557; A61P 25/18; G06F 19/28; C12Q 1/6883; A61K 31/385

(57) ABSTRACT

Methods regarding DNA methylation signature of post-traumatic stress disorder (PTSD) in brains of susceptible, resilient animals methylated in response to trauma, and S-adensoyl methionine (SAM) treated animals for deriving targets for PTSD therapeutics. Method regarding pathway analysis of the DNA methylation landscape to derive novel targets for therapeutic interventions such as retinoic acid pathway or estrogen receptor pathways. A method of treatment of PTSD comprised of Epigenetic modulators using general DNA methylation modulators such as SAM. A method of treatment of PTSD comprised of retinoic acid or vitamin A and its natural and synthetic analogs such as all-trans-retinoic acid (Tretinoin), 9-cis-retinoic acid (Alitretinoin), and 13-cis-retinoic acid (Isotretinoin) to treat PTSD. A method of treatment of PTSD comprised of a combination of Sadenosylmethionine and retinoic acid or vitamin A and its synthetic and natural analogs such as Tretinoin, Alitretinoin, and Isotretinoin.

### FIELD OF THE INVENTION

The present invention relates generally to therapeutics. More specifically, the present invention is novel therapeutics to treat post-traumatic stress disorder using DNA methylation profiles discovered in animal models.

### BACKGROUND OF THE INVENTION

Post-traumatic stress disorder (PTSD) is a trauma and stress related disorder that may develop in survivors of a traumatic event, such as a military combat. PTSD is currently defined by the coexistence of four clusters of symptoms: re-experiencing, avoidance, negative cognitions and mood and arousal, persisting for at least one month. In addition, PTSD can cause intense fear, feeling of helplessness and anhedonia¹. Fear and anxiety may increase over time, upon conditioning with stress-associated cues and in the absence of further stress exposure. Although cognitive behavioral therapy as well as pharmacotherapy with SSRIs, tricyclic anti-depressants² and cognitive enhancers in combination with psychotherapy³ alleviate PTSD symptoms, there is no reliable curative treatment to date.

A dominant brain juncture that is related to motivation and hedonia (the ability to experience pleasure or reward) is the nucleus accumbens (NAc)⁴⁻⁶. Several studies reported alterations in NAc-related functions in PTSD patients, particularly reduced reward responsivity. A decreased NAc response to reward was demonstrated in individuals suffering from common stress-related psychopathologies, such as PTSD and depression⁷⁻⁹. A recent fMRI study probed the neural correlates of sensitivity to signals of risk and reward in 24 healthy soldiers at pre-exposure and post-exposure to stressful military service, demonstrating increased amygdala activation and decreased NAc activation in response to risky anticipation and rewarding outcome, respectively^{4-8, 10-14}. Stress-induced, diminished NAc response to reward, combined with predisposed high amygdala responsivity to potential harm, may represent an underlying neural mechanism for vulnerability to stress psychopathology in humans. Neuroadaptations in the process of learning and memory have been suggested to explain the persistent distressing memories of a transient emotionally traumatic event. Since PTSD is characterized by a disordered memory, it most probably involves long-lasting changes in regulation of gene expression. This study tests the hypothesis that epigenetic alterations in the NAc play a critical role in linking traumatic exposure and presence (susceptibility) or absence (resilience) of PTSD-like behavior later in time.

Epigenetic mechanisms program the genome during cellular differentiation and embryonal development and enable a single genome to express multiple phenotypes in the different tissues that comprise a multicellular organism. Epigenetic mechanisms include covalent modification of the DNA molecule itself by methylation¹⁵ and hydroxymethylation¹⁶ as well as modifications of the histone proteins which are the building blocks around which DNA is tightly wrapped in chromatin¹⁷. DNA methylation is involved in memory formation in the adult hippocampus. For example, elevated expression of DNMT3A was implicated in memory formation in a model of reward-related associative memory¹⁸ and DNA methylation inhibitors 5-aza-2-deoxycytidine and zebularine inhibited long term potentiation¹⁹. Fear conditioning was associated with changes in methylation of candidate genes involved in memory formation²⁰. A double conditional knockout of dnmt3a and dnmt1 in adult neurons results in reduced DNA methylation and deficits in learning and memory without neuronal loss²¹. DNA methylation alterations in NAc were implicated in the reward responses to cocaine²². DNA methylation was recently examined in the NAc in an animal model of incubation of cocaine craving²³ and it was demonstrated that broad changes in DNA methylation are involved in cocaine craving and that they could be reversed by epigenetic therapeutics. Several studies in humans demonstrated associations between PTSD and differential DNA methylation. Since brain is inaccessible in humans, most studies were conducted in peripheral WBC. Using a “genome wide” approach Uddin et al., demonstrated association between PTSD and differentially methylated genes related to immune functions²⁴. Mehta et al., showed DNA methylation alterations in PTSD victims that were also abused as children in comparison with either controls or PTSD victims with no childhood trauma²⁵. Several candidate genes representing neurobiological pathways implicated in PTSD were shown to be associated with PTSD risk when differentially methylated; HPA axis (FKBP5, NR3C1), noradrenergic systems (COMT) and limbic frontal systems (SLC6A4)²⁶. Interactions between risk alleles, DNA methylation and environment were demonstrated. FKBP5 risk allele is demethylated and associated with PTSD only in people who also suffered from child adversity, thus providing a molecular mechanism for gene environment interaction in PTSD27. SLC6A3-9 repeat allele is associated with PTSD risk only when it is methylated²⁸.

A critical question that needs to be addressed is whether or not alterations in DNA methylation in response to trauma are triggered in the brain and whether or not they play a causal role in PTSD. Although risk factors for PTSD illness have been examined²⁹ resilience in adults has often been neglected³⁰. The idea that PTSD involves neuro-adaptations in the processes of learning and memory begs the question of whether resilience results from a process of protective neuro-adaptation rather than absence of neuro-maladaptation. An important question is therefore whether or not resilience is associated with a characteristic DNA methylation profile that is different from either control or susceptible individuals or whether resilience is characterized by absence of DNA methylation changes relative to control. In other words, does “resilience” reflect an active epigenetic process and a particular DNA methylation profile that plays a role in adapting the individual to PTSD? The present invention is founded on the idea that an analysis of the DNA methylation of PTSD resilient and susceptible rats in the brain will lead to critical pathways that should be targeted with therapeutics.

### SUMMARY OF THE INVENTION

In the present invention, an established rat model for post-traumatic stress disorder (PTSD) was used, which simulates several types of prevalent PTSD symptoms, including re-experiencing, avoidance, and hyperarousal³¹ after the exposure to severe trauma (predator scent). This model enables identification of animals that develop lasting symptoms which mimic the PTSD-like disorder after exposure to a traumatic experience. The animals are analyzed according to three clinically-relevant clusters of symptoms:
1. Manifestation of freezing behavior when situated alone in an open field paradigm (re-experiencing).
2. Manifestation of freezing behavior when situated with a habituated companion (social interaction).
3. Manifestation of freezing behavior after exposure to a hyperarousal event.

Only animals that show abnormal score in all three clusters, one month from the exposure to the trauma, are identified as susceptible rats (PTSD-like). This animal model allows the arrival of the following discoveries. First, DNA methylation alterations in the NAc triggered by “incubation of fear” and associate with PTSD-like behavior were discovered. Second, a unique “adaptive” DNA methylation pattern that is different from controls and the PTSD animals was discovered in resilient animals. Third, DNA methylation modulators such as the methyl donor S-adenosyl-methionine (SAM) reverse two of the three PTSD behaviors social interaction and re-experiencing and could therefore be used as a therapeutic for PTSD. Fourth, pathways that are enriched with genes that are differentially methylated in PTSD animals were discovered. Fifth, these pathways are used to identify new therapeutic targets for PTSD such as the retinoic acid or steroid antagonists and agonists. Sixth, retinoic acid reverses the hyperarousal behavior of PTSD. Seventh, a combination of retinoic acid (vitamin A) and SAM reverses all pathological behavior of PTSD animals and is therefore a new therapy for PTSD. The utility of the present invention is demonstrated that by analysis of the differentially methylated pathway, a novel combination of therapeutic agents SAM and retinoic acid (vitamin A) to treat PTSD is derived.

### BRIEF DESCRIPTION OF THE DRAWINGS

FIG. 1 is a set of diagrams depicting the rat PTSD model.

FIG. 2 is a set of diagrams depicting the differences in DNA methylation between control, PTSD susceptible and resilient animals.

FIG. 3 is a set of diagrams showing that the SAM treatment reverses two PTSD characteristic behaviors.

FIG. 4 is a set of diagrams showing that the SAM treatment alters DNA methylation patterns.

FIG. 5 is a set of diagrams depicting the correlation between differences in DNA methylation and differences in social interaction and exploration behaviors.

FIG. 6 is a set of diagrams depicting the pathway analysis of upstream regulators of genes that are differentially methylated between PTSD susceptible animals, PTSD resilient animals and controls and reversed by SAM treatment.

FIG. 7 is a set of diagrams showing that a combination of retinoic acid and SAM reverses all PTSD behaviors in PTSD susceptible animals; new therapeutic approach to PTSD.

### DETAIL DESCRIPTIONS OF THE INVENTION

All illustrations of the drawings are for the purpose of describing selected versions of the present invention and are not intended to limit the scope of the present invention.

### Embodiment 1. DNA Methylation Profiles of PTSD Susceptibility and Resilience in a Rat Model of PTSD

### Animals

Adult male Sprague-Dawley rats (250-270 gr; Harlan, Rehovot, Israel) were used and housed under conditions of constant temperature (22° C.) and 50% humidity, with a 12-h light-12-h dark cycle. The rats were allowed to habituate to the animal house for 7 days (acclimation). They were housed three per cage, two experimental rats together with a third companion rat. The same three rats remained together until the end of the study. Food and water were provided ad libitum. All experiments were performed between 08:00 and 14:00 in daylight. All animal procedures were approved by the Bar-Ilan University Animal Care Committee and were carried out in accordance with the NIH Guide for the Care and Use of Laboratory Animals.

### Behavioral Procedure

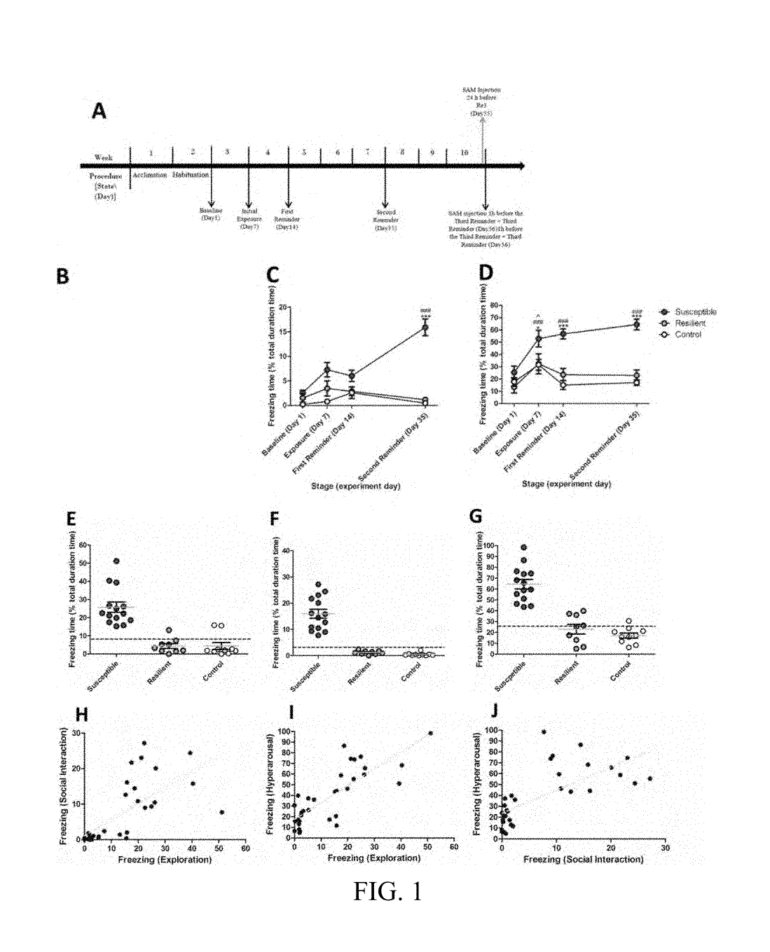

The model used in this research is based on Kesner et al., 2009³². This model consists of several stages encompassing 10 weeks (56 days), detailed below and depicted in FIG. 1. The animals are analyzed according to three clinically-relevant clusters of symptoms: manifestation of freezing behavior when (a) situated alone in an open field paradigm (re-experiencing), (b) situated with a habituated companion (social interaction) and (c) after exposure to a hyperarousal event³³. The range of each measured behavioral procedure was determined for all animals after the base-line (week 3). The data collected at baseline was analyzed and the explore procedure was applied using SPSS 11 to define the range of the population. The upper and lower levels of this range were treated as ‘normal baseline’ and alterations from this range were used to define PTSD-like behavior (susceptible). Animals were subsequently divided into two groups: susceptible (PTSD-like rats exhibiting behavior above ‘normal baseline’ in all three conditions) and resilient (non-PTSD-like rats exhibiting at least one behavior in the ‘normal baseline’ range)³².

### Habituation

Each experimental or naive rat was habituated for 7 days to their home cage (the first week of the habituation). For the first stage of the experiment each experimental or naive rat was habituated for 7 days to an open field apparatus together with a companion rat from their home cage, 5 min per day for 7 days (the second week of the experiment). The open field apparatus was a 90×90×30 cm Plexiglas box, which was placed under a camera. The video and computer equipment were situated in a separate room where all video and observation analyses were performed.

### Baseline

After 14 days (Day 1) of habituation, baseline freezing levels of experimental and naive rats were measured prior to stress exposure. Behavioral parameters and measurements are described below (Behavioral Measurements section).

### Initial Exposure

One week later (Day 7), experimental rats were exposed to stress, i.e. predator scent. Individual rats were placed for 30 min in a clean plastic cylinder (diameter: 30 cm, height: 33 cm) containing 125 ml of well-soiled cat litter, used by a cat during the 24 h prior to the experiment. After exposure to stress, each rat was transferred to the open field, and three behavioral parameters were measured (as described in the Behavioral Measurements section below). The cylinder was cleaned between testing of each rat. Temperature and humidity conditions in the stress exposure area were identical to those in the housing cages and open field. The companion rats were not exposed to stress, and were housed in separate cages in a separate housing area during all experiments. The same cat was used for the litter introduced during initial exposure.

### First Reminder (Re1)

One week later (Day 14), rats were re-exposed for 30 minutes to litter with the same texture, but without the cat scent. Behavioral parameters were subsequently tested (as described below).

### Second Reminder (Re2)

Three weeks later (Day 35), rats were again exposed for 30 min to litter with the same texture and without cat scent. Behavioral parameters were subsequently tested (as described below). Behavioral data for this testing stage were analyzed according to the division criteria detailed below. Based on the obtained results, experimental rats were defined as either susceptible or resilient.

### Behavioral Measurements

After exposure to cat litter, each rat was placed in the open field for measurement of PTSD-like behavior. In the open field, each rat was consecutively tested under three behavioral conditions: alone (termed “exploration”; 5 min), with their habituated companion (“social interaction”; 5 min), and then during post-startle response after exposure to a loud noise (“hyperarousal”; 5 min). The loud noise was a 36.57±0.3 dB pick/scale A over baseline noise of 55±0.5 dB measured by Quest instrument, Quest Technologies, model 2900, calibrated by QC 10 calibrator 114 dB at 1000 Hz, which was sounded to the tested rats during the first 5 min of hyperarousal. The following behavioral parameter was measured during each of the three conditions: freezing (amount of time that the animal remained completely immobile, for at least 2 s). The tested rats were not removed from the open field nor touched during the three testing conditions. Behavioral parameters were videotaped and monitored using Observer apparatus and software (Noldus, The Netherlands).

### Behavioral Data Analysis: Criteria for Subdivision to Susceptible and Resilient Groups

Baseline behavioral data was analyzed by the Explore procedure in SPSS 11 (IBM Software), to define the interquartile range of the population for each behavioral parameter. Results inside the upper range and lower range limits were treated as ‘normal baseline’. Alterations from this range were used to retrospectively define extreme behavior (after Re2 testing was concluded). According to the determined range, animals were divided into two sets: ‘susceptible’ (exhibiting PTSD-like behavior above ‘normal baseline’ in all three conditions) and ‘resilient’ (animals exhibiting at least one behavior in the ‘normal baseline’ range) (1).

### Statistical Analysis of Behavior

Exploration, social interaction and hyperarousal data were analyzed by a two-way analysis of variance (ANOVA) (with a Bonferroni correction), followed-up by one-way ANOVAs with repeated measures followed by Bonfferoni post-hoc test to compare the different time points in each group (susceptible vs. resilient vs. control) during each behavioral condition (exploration, social interaction and hyperarousal). For analysis of PTSD-like behavior in rats treated with SAMe, a two-way ANOVA with repeated measures with Bonfferoni correction. p<0.05 was considered significant.

### PTSD Like Behaviors and Resilience Following Incubation of Fear in Rats Exposed to Trauma

A total of 50 Sprague-Dawley rats were exposed to the traumatic event (litter with cat odor) and stress-associated reminders. Posttraumatic stress disorder-like behavior of each animal was compared both with its own baseline and with the range in the population to ensure that two unambiguous subpopulations “resilient” and “susceptible” are identified. The incidence of susceptible rats was approximately 20% (14 susceptible and 26 resilient animals). 10 animal were used as a control group (did not go under the cat odor exposure). Analysis of all baseline behavioral samples revealed that the upper level for excluding outliers (95% confidence) in the exploration, social interaction and hyperarousal conditions was twice the interquartile range. Animals were separated by 3 behavioral tests (FIG. 1B-D), that showed high correlation within themselves (FIG. 1E-G). Exploration, social interaction and hyperarousal data was analyzed by a two-way analysis of variance (ANOVA) (with a Bonferroni's correction), followed-up by one-way ANOVAs with repeated measures followed by Bonferroni's post hoc test to compare the different time points in each group (susceptible, resilient and control) during each behavioral condition (exploration, social interaction and hyperarousal). During the ‘exploration’ test, a two-way analysis of variance (ANOVA) comparing freezing behavior over the four time points (within subjects) and the three groups (susceptible, resilient and control) revealed a main effect of group (F[2,90]=25.75; p<0.0001), main effect of time (F[3,90]=8.46; p<0.0001) and main effect of interaction (F[6,90]=7.79; p<0.0001). Bonferroni's post hoc correction showed significant differences in freezing behavior of susceptible group versus resilient and control groups at Re-1 and Re-2 time points. (***p<0.01) (FIG. 1B). During the ‘social interaction’ test, a two-way analysis of variance (ANOVA) comparing freezing behavior over the four time points (within subjects) and the three groups (susceptible, resilient and control) revealed a main effect of group (F[2,90]=41.55; p<0.0001), main effect of time (F[3,90]=8.14; p<0.0001) and main effect of interaction (F[6,90]=11.72; p<0.0001). Bonferroni's post hoc correction showed a significant difference in freezing behavior of susceptible group versus resilient and control groups at the Re-2 (***p<0.0001) (FIG. 1C). During the ‘hyperarousal’ test, a two-way analysis of variance (ANOVA) comparing freezing behavior over the four time points (within subjects) and the three groups (susceptible, resilient and control groups) revealed a main effect of group (F[2,90]=24.29; p<0.0001), main effect of time (F[3,90]=11.09; p<0.0001) and main effect of interaction (F[6,90]=5.40; p>0.0001). Bonferroni's post hoc correction showed a significant difference in freezing behavior of susceptible group versus resilient and control groups at exposure, Re-1 and Re-2 time points (*p<0.05, ***p<0.0001, ***p<0.0001 respectively) (FIG. 1D). The group time point interaction reveals that beyond the particular test, the susceptible group increased its freezing duration over time, significantly more than the resilient and the control groups. During the ‘exploration’ test, freezing behavior of susceptible group was significantly elevated from baseline after Re-1 and after Re-2 (one-way ANOVA: F[3, 13]=18.85; p<0.001; Bonferroni's post hoc: ###p<0.0001). For resilient and control groups, no significant differences were detected (p>0.05) (FIG. 1B). During ‘social interaction’ test, freezing behavior of susceptible group after Re-2 were significantly elevated from baseline (one-way control rats, no significant differences were detected (p>0.05) (FIG. 1C). During ‘hyperarousal’ test, freezing behavior of susceptible group after exposure, Re-1 and Re-2 were significantly elevated from baseline (one-way ANOVA: F[3, 13]=13.98; p<0.0001; Bonferroni's post hoc: ###p<0.0001). For resilient group, no significant differences were detected (p>0.05). For control group, freezing behavior after the exposure was significantly elevated from baseline (one-way ANOVA: F[3, 9]=7.15; p<0.01; Bonferroni's post hoc: ̂p<0.05) (FIG. 1D). The entire distribution of freezing data is presented for each of the three behavioral conditions at the Re-2 time point (FIG. 1E-G). FIG. 1E-G reveals a greater increase from baseline in PTSD-like behavior of the susceptible group, for exploration (FIG. 1 E), social interaction (FIG. 1F) and hyperarousal (FIG. 1G) tests, as compared with resilient and control rats. The distribution data for the Re-2 time point, reveals an increase from baseline in PTSD-like behavior of the susceptible group, for all three behavioral tests, as compared with resilient and control rats. Pearson product-moment correlation between exploration with social interaction (FIG. 1H), exploration with hyperarousal (FIG. 1I) and social interaction with hyperarousal (FIG. 1J) were r=0.69, 0.80 and 0.70; p<0.0001 respectively.

### Tissue Collection

Brains were removed immediately after exposure to litter with cat scent immediately after Re3. Each brain was placed in a Perspex brain matrix and sliced into 1.0 mm segments. The NAc was punched out using a micro dissecting needle of 13-14 G. Brain tissue was frozen at −70° C. until DNA and RNA extraction.

### Extraction of DNA

Genomic DNA was extracted using All Prep DNA/RNA Micro Kit, Qiagen (Hilden, Germany)

### Capture Bisulfite Sequencing and DNA Methylation Mapping

The SeqCap Epi Enrichment System was used for target bisulfite sequencing of promoters and enhancers in the rat genome. The rat (rn4) target probes were custom designed by utilizing H3K4me1 and H3K4me3 (indicating enhancers and promoters respectively) signals from mouse samples as there is no currently available ChIP data of H3K4me1 and H3K4me3 with respect to the rat species. PuttivH3K4me1 and me3 were selected by mapping and aligning genomic fragments from mouse to the rat and retaining any fragments that share a high similarity and the subsequent associated signals. A total of 53374 target probes have been designed spanning 48072248 base pairs of sequence with a mean probe size of 900 bp. Biotinylated target probes were designed for both strands of bisulfite converted genomic DNA. Bisulfite treated genomic DNA is ligated to methylated NGS adaptors, hybridized to the biotinylated oligonucleotide probes which is followed by a series of washes of off-target DNA sequences and unbound DNA. The resulting isolated specific DNA then undergoes PCR amplification for downstream sequencing on Illumina Hiseq 2000 sequencer. Capture and hybridization is performed as a service for fee by ICRM in Montreal licensed by Roche/Nimblegen. The following bioinformatics workflow is used in the lab. Quality control is done using FastQC to assess sequencing scores and other quality metrics. If necessary, trimming of raw file is performed using Trimmomatic to remove low scoring sequencing results and/or adapter contents. Sequences are aligned to rn4 rat reference genome using Bsmap v2.89. A typical alignment rate of 90%-95% is achieved (30× coverage). The resulting sequencing alignment file (SAM) is converted to binary alignment format (BAM) for downstream analysis and filtering. BAM files are split, merged, and sorted by strand for additional downstream filtering and analysis. Duplicate reads within the BAM files are removed via Picard Tools (v1.93, BROAD institute) strand-specifically and the separate BAM files are merged back together and examined by Bamtools to assess whether the sequences are properly mapped to genome, whether both paired end reads exist (for paired end sequencing), and whether paired end reads are properly paired to each other (for paired end sequencing). Using the final BAM file, methylation ratios and coverage is recovered by provided scripts within Bsmap as well as methylation context. Annotation of genomic locations is performed by HOMER. Computation of differential methylation sites and tiled windows of differentially methylated regions was performed using a logistic regression method with the Methylkit R package in Bioconductor1. FDR adjusted P value significance threshold is held at <0.2.

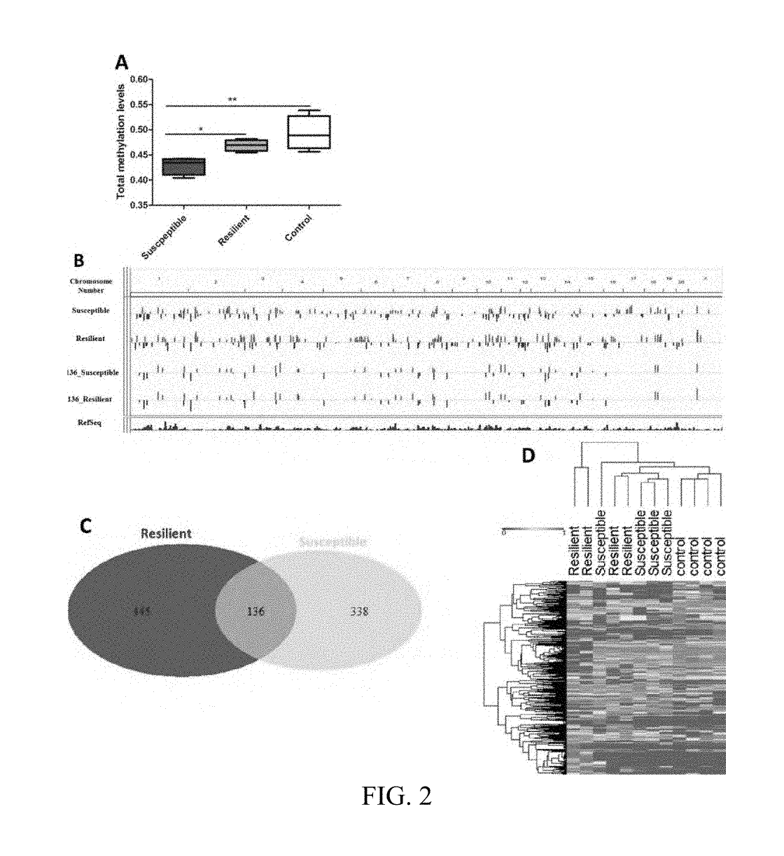

Whether trauma triggers a distinct DNA methylation profile in the NAc that differentiates traumatized animals from controls, whether resilient or susceptible animals exhibit DNA methylation responses to trauma and whether these responses are different between the resilient and susceptible groups were tested. NAcs were isolated from the animals (n=4-7 per group) following the third reminder and DNA and RNA were extracted as described in the methods. DNA was bisulfite converted, enriched using capture arrays for all predicted promoters and enhancers in the rat genome and was then subjected to next generation sequencing. The distribution of DNA methylation levels across the captured regions as described was first mapped. On average 5306667 CGs per animal was sequenced which showed an average coverage per CG of 39. The question of whether exposure to trauma triggered changes in the DNA methylation profile in the susceptible and the resilient groups was addressed by computing differential methylation sites using a logistic regression method with the Methylkit R package in Bioconductor1. First, the total CG methylation levels along the susceptible, resilient and control groups were compared. A significant methylation differences between the susceptible group to the resilient and control groups was revealed, on average the susceptible group is hypo-methylated in comparison with either the resilient or control groups (FIG. 2A) (One-way ANOVA: F[2, 9]=8.01; p<0.05; Newman-Keuls Multiple Comparison Test: *p<0.01 and **p<0.001). Afterward the susceptible group (saline treated) and the control group (saline treated) were compared. The data demonstrates 263 CGs that were de-methylated and 211 CGS that were hyper-methylated at a threshold of delta>10—and Q value of 0.2 (FIG. 2B). Resilience is characterized by absence of PTSD-like behavior on the long term (experiment day 35) in spite of similar exposure to the trauma and its reminder. The question of whether the resilient animals escaped the DNA methylation response to trauma and will exhibit no differences from control or whether a non-PTSD-like behavior evoked methylation alterations that might have been involved in a mechanism of adaptation was addressed. The DNA methylation state in resilient group (saline treated) with the control group (saline treated) was compared which revealed 275 CGs de-methylated and 306 CGs hyper-methylated at a threshold of >10; and Q value of 0.2 (FIG. 2B). Then, whether similar genes are affected in resilient and susceptible groups and whether the changes occur in the same or different directions was tested. Comparing the lists of differentially methylated CGs between the two groups that were exposed to the same ‘incubation of fear’ model, revealed that 136 CGs sites overlapped between the two groups (p=2.721E-272 FISCHER's EXACT two-tailed TEST). These mutual sites were in the same direction of methylation which raises the possibility that these CGs sites may represent the ‘trauma sites’, related to the traumatic exposure itself and not to the development of susceptibility or resilience (FIG. 2B-C). It is hypothesized that the remaining CG sites that were different between resilient and control groups represent the DNA methylation profile involved in resilience.

Hierarchical clustering by One minus Pearson correlation of the 738 (excluding the ‘trauma sites’) CGs sites that were altered in response to the ‘incubation of fear’ model in either the resilient or susceptible groups revealed that these sites cluster the resilient susceptible and control animals by their DNA methylation profile. (FIG. 2D). The data show that the incubation of the traumatic outcome elicits a DNA methylation alteration in all exposed animals, albeit differently in resilient and susceptible animals. Resilient animals' DNA methylation profile is different from both control and susceptible animals and although overall their behavior is normal, the associated DNA methylation is different. This is the first demonstration of a DNA methylation profile of “resilience”. Ingenuity Pathway Analysis (IPA) analysis of canonical pathways revealed that the differential methylated CGs that were changed in the resilient and susceptible groups have strong relevance to PTSD. Such pathways are: glucocorticoid signaling, corticotrophin releasing hormone signaling, serotonin and dopamine degradation, AMPG signaling, ERK/MAPK signaling, GDNF family ligand-receptor signaling, axonal guidance signaling, synaptic long term depression and potentiation, CREB signaling in neurons, Dopamine-DARPP32 Feedback in cAMP Signaling, cAMP-mediated signaling, protein kinase A signaling and more.

### Utility of DNA Methylation Profiles of PTSD Resilience and Susceptibility Taught by the Present Invention

The pathways taught by the present invention serve as platform for discovery of new drugs to treat PTSD. Each of the pathways discovered here are targets for therapeutic interventions to convert PTSD susceptible patients to become either PTSD or resilient or better indistinguishable from controls. The broad changes in DNA methylation instructed that treatment should be done with a broad DNA methylation modulator.

### Embodiment 2. Reversal of Two PTSD Behaviors, Exploration and Social Interaction by SAM Treatment

The first embodiment of the present invention reveals a broad change in DNA methylation in susceptible animals, it was reasoned therefore that agents that affect DNA methylation might reverse the PTSD-like phenotype. The susceptible animals with the ubiquitous methyl donor SAM were treated which is an approved natural supplement with a good safety record that could be translated to humans.

After Re2, susceptible, resilient and control group were intraperitoneally injected with S-adenosylmethionine (SAM) (Life Science Laboratories Lakewood N.J.) or saline (25 mk/kg), 24 hours and 1 hour before the third reminder. Each group was randomly divided into two groups, each receiving an injection of SAM or saline intraperitoneally. Afterward the third reminder was performed and rats were again exposed for 30 min to litter with the same texture and without cat scent. Behavioral parameters were subsequently tested (as described below).

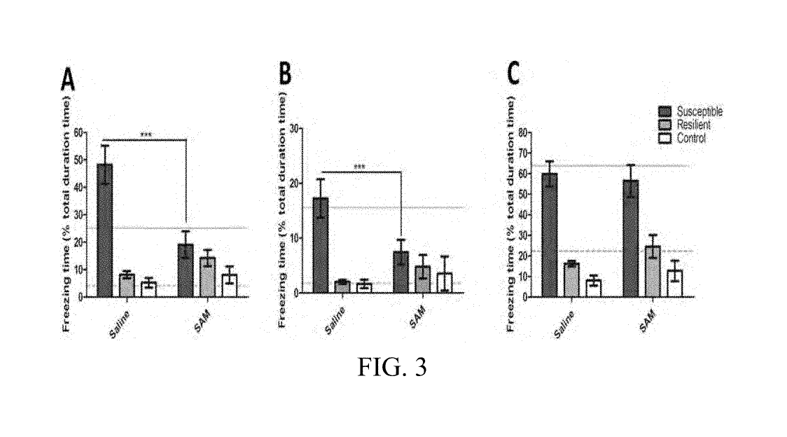

Each subpopulation of susceptible, resilient and control groups (see methods) received either SAM (25 mg/kg) or saline (i.p., n=4-7 per group), 24 h and again 1 h before the third reminder (Re-3). SAM treatment significantly attenuated freezing behavior in two of three behavioral tests (‘exploration’ and ‘social interaction’ tests) in the susceptible group in comparison with the group that had been treated with saline (FIG. 3A-C). More specifically, during the ‘exploration’ test in Re-3 time point (FIG. 3A), freezing behavior of susceptible-SAM treated rats was significantly decreased from freezing levels of susceptible-saline treated rats and no significant differences were observed between susceptible-SAM treated rats to the resilient and control treated with SAM or saline (one-way ANOVA: F[5, 27]=13.07; Bonferroni's post hoc: ***p<0.0001). During the ‘social interaction’ test in Re-3 time point (FIG. 3B), freezing behavior of susceptible-SAM treated animals was significantly decreased from freezing levels of susceptible-saline treated rats and no significant differences were observed between susceptible-SAM treated rats to the resilient and control treated with SAM or saline (one-way ANOVA: F[5, 26]=7.722; Bonferroni's post hoc: ***p=0.0001). During the ‘hyperarousal’ test in Re-3 time point (FIG. 3C), there was no difference in freezing behavior between susceptible-SAM treated rats and susceptible-saline treated rats but both susceptible-SAM and susceptible-saline treated rats were different from the resilient and control treated with SAM or saline (one-way ANOVA: F[5,27]=16.75; Bonferroni's post hoc: ***p<0.0001). The fold change in freezing behavior in ‘exploration’ and ‘social interaction’ tests (from Re-2 to Re-3) between the susceptible-treated rats (FIG. 3D-G) indicates that SAM was effective in reducing these behaviors while it was ineffective in changing freezing behavior in the ‘hyperarousal’ test (from Re-2 to Re-3) (FIG. 3H-I). These results show that SAM attenuated two of the three PTSD-like behaviors in the susceptible animals.

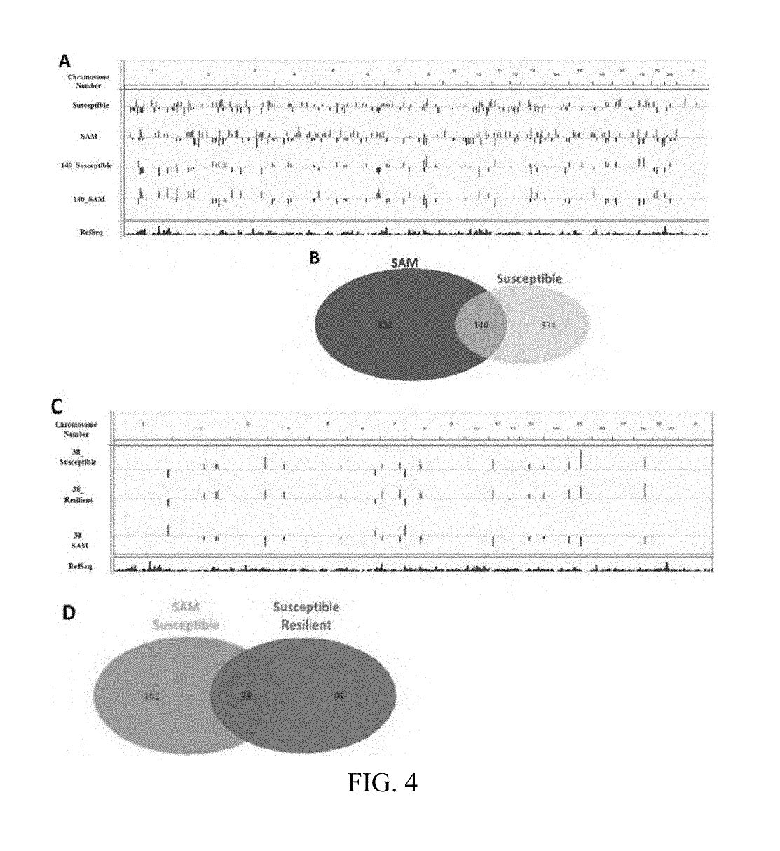

SAM treatment alters the DNA methylation profile of susceptible animals. If the mechanism of action of SAM on PTSD-like behaviors is mediated by DNA methylation, DNA methylation profiles that are characteristic of the susceptible state should be effected by the treatment. The DNA methylation pattern of susceptible-SAM treated animals was first compared with susceptible-saline treated rats, which revealed 599 CGs sites that were de-methylated and 363 CGs sites that were hyper-methylated at a threshold >10; and Q value of 0.2 (FIG. 4A). Susceptible-saline and susceptible-SAM treated animals comparison delineated 140 CGs sites (p=1.561E-321FISHER's EXACT two-tailed TEST) that were differentially methylated in the susceptible animals and were then reversed by the SAM treatment (FIG. 4A-B). These data are consistent with the hypothesis that SAM reversed a fraction of DNA methylation changes (CG sites) induced in the susceptible animals, and that this partly or fully mediates the therapeutic effects of SAM.

SAM also affected 38 CG sites that were differentially methylated in both the susceptible and the resilient rats (FIG. 4C-D). In summary, SAM's treatment reversed a significant fraction of DNA methylation sites that were altered in susceptible rats relative to controls (140 CG sites, 30% of total susceptible sites) as well as sites that are altered in both the resilient and susceptible animals.

Quantitative distribution of DNA methylation across individual rats correlates with the quantitative distribution of the behavioral phenotypes and is shifted with SAM treatment.

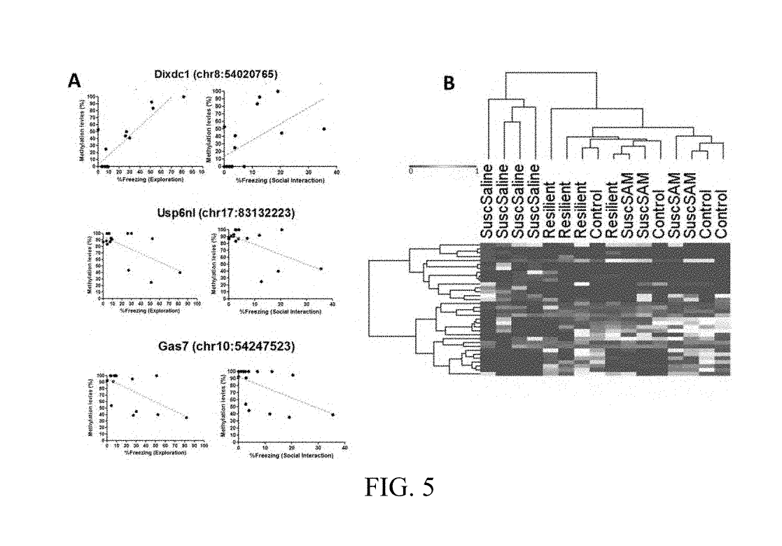

The three behavioral phenotypes are quantitatively distributed across individual animals. DNA methylation states are also quantitatively distributed amongst the individual animals. Therefore, it was hypothesized that the quantitative distribution of these phenotypes correlates with the quantitative distribution of DNA methylation levels of particular sites in the NAc. The hypothesis was tested by performing a Pearson correlation between the level of methylation of 140 CG sites that were found to be differentially methylated between the susceptible animals and the SAMe's treated animals in the three behavioral tests: exploration, social interaction and hyperarousal across the individual animals whose DNA methylation levels were mapped (n=16) at a threshold of r>0.5 and adjusted p value <0.05. 23 CG sites that were reversed by SAM's treatment exhibited linear correlation with exploration phenotype and 21 CG sites exhibited linear correlation with social interaction phenotype and only 9 CG sites are in common to the exploration and social interaction behavioral tests (FIG. 5A). Hierarchical clustering by One minus Pearson correlation of the correlated CGs sites that were altered in response to SAM treatment along the experimental groups presented in FIG. 5B, reveals that these sites cluster the susceptible-saline treated group separately from the resilient, susceptible-SAM treated and control groups by their DNA methylation profile.

These data support the idea that inter-individual quantitative differences in behavior are associated with quantitative differences in DNA methylation and that SAM shifts the distribution of methylation of these sites and the correlated behaviors.

Importantly, none of the sites that were correlated with the hyperarousal behavior were altered with SAM which is consistent with the observation that SAM treatment does not reverse the hyperarousal behavior, suggesting that the hyperarousal behavior is associated with different DNA methylation profiles than the other two behaviors.

### Utility of the Discovery

The discovery could be translated to humans into a new therapy of PTSD using SAM.

### Embodiment 3. Discovery of “Upstream Regulators” of Genes that are Differentially Methylated in Susceptible and Resilient Traumatized Animals and Reversed by SAM Treatment

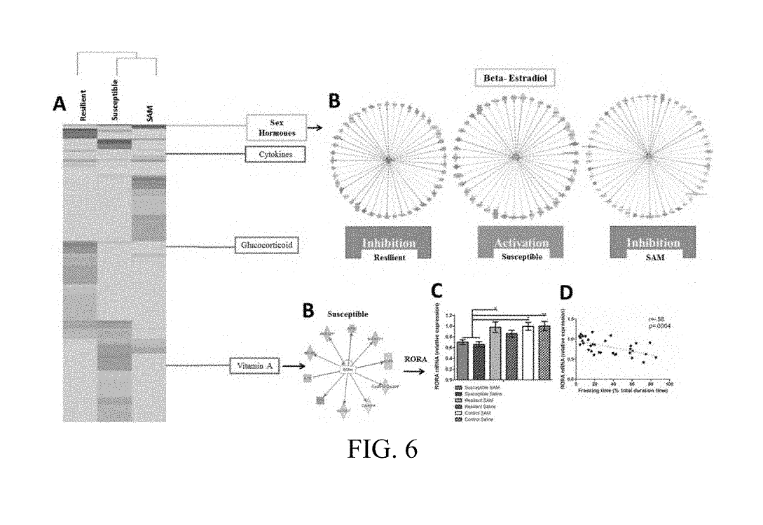

If DNA methylation plays a causal role in PTSD, comparative analysis of upstream regulators of genes whose DNA methylation is different in control, susceptible, resilient and SAM treated animals could reveal important putative new pathways and networks involved in PTSD molecular pathology that are candidates for therapeutic intervention (FIG. 6A). For example, the Beta estradiol downstream pathway is upregulated (activated) in the susceptible group and downregulated (inhibited) in the resilient group and is also inhibited by SAMe's treatment (FIG. 6B), pointing to estradiol downregulation as a target for relieving PTSD-like symptoms. The involvement of estradiol responsive genes suggests also an important role for female sex hormones in determining susceptibility to PTSD³⁴. Other up-regulators of differentially methylated genes are presented in the Heatmap. These pathways are relevant to anxiety and memory pathways such as: sex hormones, glucocorticoid and different cytokines.

The retinoic biosynthesis and degradation pathway was also enriched with differentially methylated genes. In the susceptible group there was an enrichment in downstream targets of the RAR-Related Orphan Receptor A (RORA), however the impact on level of activity of this pathway could not be derived from this analysis (FIG. 6B). Therefore, the levels of RORA mRNA expression in the different groups was first determined. RORA mRNA expression levels were higher in either control or resilient animals treated with either saline or SAM than in susceptible animals (*p<0.05, *p<0.05 and **p<0.01 respectively) (FIG. 6C), consistent with the possibility that this gene is involved in the pathology of PTSD. Interestingly, SAM had no effect on the level of expression of RORA. Therefore, it was hypothesized that the RORA pathway is involved in the hyperarousal behavior which is resistant to SAM's treatment and characteristic of the susceptible PTSD group. The correlation between the mRNA expression of RORA and the three behavioral tests was first measured; as hypothesized, only the hyperarousal behavior was correlated with the mRNA expression of RORA (FIG. 6D). This instructs that treatment with retinoic acid would reverse the third PTSD behavior that is not reversed with SAM treatment.

Utility of Discovery of Pathways Regulating Genes that are Differentially Methylated in PTSD

The pathways discovered here will be utilized for the discovery of new drug targets for treating PTSD such as estrogen receptor agonists or antagonist and retinoic acid.

### Embodiment 4. Reversal of All Three Characteristic Pathological Behaviors of PTSD with a Novel Combination of Retinoic Acid and SAM

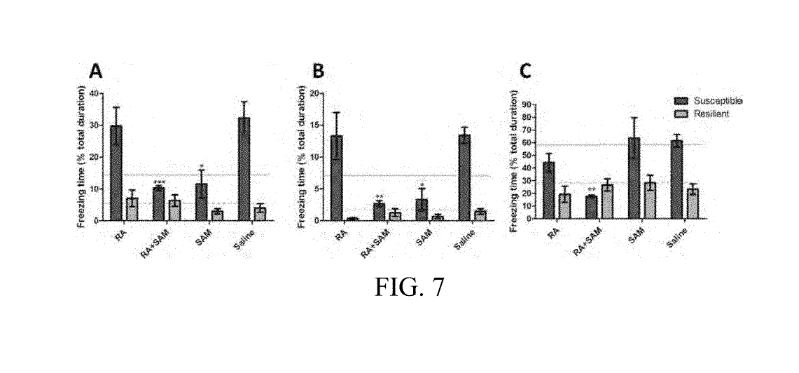

Since it was discovered that retinoic acid reverses the third pathological behavior that is not reversed by SAM, whether stimulating this pathway by supplying retinoic acid will reverse PTSD “hyperarousal” feature of the PTSD pathology and whether a combination of SAM and vitamin A (retinoic acid (RA)) will have a synergistic effect on PTSD-like behavior, relieving all three PTSD symptoms, was tested. Each subpopulation of susceptible and resilient group received either SAM (25 mg/kg), retinoic acid (2 mg/kg) or saline (i.p., n=3-6 per group) twice, 24 h and 1 h before the third reminder (Re-3). It was found that the combination of SAMe and RA significantly attenuated freezing behavior in all of the three behavioral tests in the susceptible group in comparison with the groups that were not treated with this ‘cocktail’ (FIG. 7A-C). More specifically, during the ‘exploration’ test in Re-3 time point (FIG. 7A), freezing behavior of susceptible-SAM+RA and susceptible-SAMe treated animals was significantly decreased from freezing levels of susceptible-RA and susceptible-saline treated animals and no significant differences were observed between them and between the resilient treated animals with SAM, RA or saline (one-way ANOVA: F[7, 26]=11.81; Bonferroni's post hoc: *p<0.05, ***p<0.0001). During the ‘social interaction’ test in Re-3 time point (FIG. 7B), freezing behavior of susceptible-SAM+RA and susceptible-SAMe treated animals was significantly decreased from freezing levels of susceptible-RA and susceptible-saline treated animals and no significant differences were observed between them and between the resilient treated animals with SAM, RA or saline (one-way ANOVA: F[7, 27]=10.60; Bonferroni's post hoc: *p<0.05, **p<0.01). During the ‘hyperarousal’ test in Re-3 time point (FIG. 7C), freezing behavior of susceptible-SAM+RA treated animals was significantly decreased from freezing levels of susceptible-SAM and susceptible-saline treated animals and no significant differences were observed between susceptible-RA and between the resilient treated animals with SAM, RA or saline (one-way ANOVA: F[7, 27]=7.03; Bonferroni's post hoc: **p<0.01). This is an attractive therapeutic strategy as both compounds are approved nutritional supplement. The results presented in FIG. 7 demonstrate that RA supplementation inhibits the hyperarousal phenotype of susceptible animals, but this effect is enhanced when RA is combined with SAM. These results support the hypothesis and illustrate the value of DNA methylation profiling in combination with an epigenetic modulator. These data are also consistent with the hypothesis that alterations in DNA methylation play a causal role in PTSD.

### Applications of the Present Invention

The main application of the present invention is in the field of PTSD therapeutics and drug discovery. The DNA methylation profiles and pathways that were discovered provide a general platform for discovery of new drugs to treat PTSD. Moreover, two agents SAM and retinoic acid that in combination reverse PTSD and could be used in PTSD therapeutics are disclosed.

Epigenetic mechanisms offer an attractive hypothesis for explaining the lasting effects of transient exposure to trauma as well as the potential impact of life adversity and gene environment interactions. However, evidence was lacking regarding DNA methylation alterations in brain regions known to be involved in PTSD and there was no evidence of a causal relationship between epigenetic alterations and PTSD behaviors. Addressing these questions is critical for understanding the disease as well as for prevention and intervention. These questions could only be addressed using a reliable animal model with construct validity. Previously, it was reported on such an animal model that mimics PTSD³². This model demonstrates a progressive increase in fear behavior and a shift to anxiety over time to the stress itself and to its reminders in the susceptible group (PTSD-like rats).

The findings show a time-dependent gradual intensification of fear response to recurring stress reminders over 35 days. The susceptible group's behavior corresponds with the clinical definition of PTSD in the human population, including anxiety behavior, social deficit and avoidance behavior and high levels of arousal behavior^{30,36}. In addition, exogenous stressors, such as exposure to a live predator^{26,37}, psychological stress³⁷ and the predatory cue used in this research correspond with natural stressors. One of the salient characteristics of PTSD is resilience; the distribution of PTSD among trauma's victims is 5%-30%, whereas other that were exposed to the same trauma appear to be resilient to it²⁷. Therefore, understanding the mechanisms underpinning PTSD necessitates accounting for resilience and a model that has construct validity should exhibit partial resilience as well. This also highlights both importance of individual analysis of behavior and comparison of each exposed animal both with its own baseline and with the entire population. Although this analysis yields a lower percentage of susceptible rats, it more accurately simulates the heterogeneous pattern of response to stressful events in the human population. Thus, the combination of the model components, i.e., exogenous stimuli, simulation of clinical manifestation, and individual categorization, can produce more reliable results. Despite the increased risk that is associated with trauma, some individuals develop strong coping responses which defines them as a resilient group^{38, 39}. The ability to cope adaptively with trauma refers to the resilient characteristic⁴⁰. Resilience characteristics are likely to attenuate risks of developing PTSD, perhaps through effective emotional regulation, tolerance of negative affect, or active seeking of supportive or nurturing relationships⁴¹. Although detailed information is available on risk factors for posttraumatic stress disorder^{29,42}, resilience in adults has often been misunderstood because much of what is known about coping in trauma comes from studies of treatment seeking or distressed individuals recruited from psychiatric treatment settings⁴³.

Since PTSD is characterized by a disordered memory⁴⁴, understanding long-lasting changes in the programming of gene expression may help elucidate the factors that are involved in embedding the traumatic exposure in neural circuits well as the separation of susceptible from the resilient individuals. DNA methylation is a chemical modification of DNA that plays an important role in defining stable long lasting gene expression programs during differentiation^{2,3} and is also implicated in mediating the long term effects of early life adversity⁴. Moreover, DNA methylation varies between individuals even on the same genetic background and might provide an explanation for the inter-individual variation in the response to past traumas⁵. In the current discovery, the above mentioned model was used to examine at a single nucleotide resolution the known functional regulators of transcription in the rat genome, promoters 5′UTR, 3′UTR and enhancers using capture bisulfite sequencing. It was hypothesized that if changes in DNA methylation occur, they will not be limited to a small list of candidate genes but will involve several genes in functional gene pathways. Genes do not act independently but in complex networks and it stands to reason that complex behaviors involve alterations in complex neuronal circuits and in complex cellular pathways of gene expression. It also stands to reason that reversing a PTSD-like phenotype, for example, will be possible if pathways rather than single genes are targeted. Using an unbiased comprehensive approach provides an opportunity to characterize pathways that are affected in PTSD as well as those that confer resilience that could serve as new candidates for therapeutic interventions. The focus was on NAc since it is a central region in the limbic system known to mediate reward, motivation and hedonia⁴⁻⁶. As for PTSD, anhedonia is one of its main characteristics⁴⁵, albeit less studied. Recent findings have demonstrated that the NAc may have a significant role in PTSD progress. Moreover, broad changes in DNA methylation occur in the NAc during incubation of cocaine craving supporting the hypothesis that DNA methylation might be also critical in incubation of fear in the NAc²³.

First, it is shown that despite the risk to develop PTSD after a traumatic event in the animal model, the resilient rats are likely to attenuate the PTSD-like symptoms and are likely to assuage the expression of PTSD-like symptoms and exhibit behaviors similar to the control group, as in humans. This means that resilient rats are not unaffected subjects but rather individuals that develop resistance.

Second, it is shown that susceptible animals show differences in DNA methylation in the NAc as compared to control animals that involve, as hypothesized, a number of functional pathways such as neurological and psychological disorders, cellular function maintenance, anxiety, learning and memory and more. In total, 474 CG sites were differentially methylated from the control group in the susceptible group and they are associated with 379 different genes. These pathways serve as a platform for discovery of new drug targets to treat PTSD.

Third, it is shown that resilient animals also exhibit differentially methylated CG sites from control animals and these partially overlap with the susceptible DNA methylated differences. These changes occurred in the same direction of methylation (the “trauma sites”), but the other methylated CG sites of the resilient group are unique to this group. Here, the first evidence that resilience is associated with an active DNA methylation response rather than absence of a change in DNA methylation is provided. This suggests that DNA methylation might be involved in resilience phenotype. In total 581 CGs were DNA methylated in the resilient group and they are included in 475 different genes. In addition, the resilient group exhibit DNA methylation differences from the control group which were enriched in a number of functional pathways such as nervous system development and function, psychological disorders, learning and memory, anxiety, cell-to-cell interaction signaling and more. It is tempting to speculate that trauma evokes a heterogeneous continuous DNA methylation response that is potentially protective but could become maladaptive when it fails to target the complete “protective” DNA methylation program.

Examining the individual behavioral phenotypes of the animals in the study reveals that it shows a continuous inter-individual variation resembling a quantitative trait with a threshold rather than a binary trait. While classical genetics can easily explain binary traits caused by Mendelian inheritance of rare alleles, the continuous distribution of quantitative traits is more difficult to explain. The state of methylation of genes could vary from cell to cell even in the same tissue increasing the potential for a continuous variation. It is recently shown that quantitative distribution of DNA methylation in the epithelial growth factor receptor promoter (Egfr) is associated with quantitative distribution in body size in ants⁴⁶. Quantitative distribution of DNA methylation is thus well poised to explain the quantitative inter-individual variations in the three behavioral traits that characterize PTSD. It is shown here that quantitative individual variations in each of the three behaviors exploration, social interaction and hyperarousal correlate with individual variation in 43 differential DNA methylated sites that were affected by SAM's treatment. Interestingly, two of the behaviors show an overlap in 9 differentially methylated CGs while hyperarousal seems to be controlled by an independent set of CGs. This is consistent with the fact that SAM's treatment does not affect hyperarousal nor does it change the state of methylation of sites that correlate with this behavior.

The data instructs first on the nature of changes in the DNA methylation landscape following ‘incubation of fear’ in NAc of resilient and susceptible animals as well as on the downstream pathways that are targeted by these processes. The wide cast of genes that are altered suggest that reversal of PTSD will probably require approaches that result in broad modulation of the DNA methylation agents and in modulating critical cellular pathways rather than single proteins or single targets of methylation. The ubiquitous methyl donor SAM was tested although it is clearly nonspecific for particular genes or group of genes and is involved in methylation of other macromolecules including histones. It was previously shown that SAM could cause broad but nevertheless selective alteration of DNA methylation in cancer cells⁶ as well as the brain⁷. SAM is particularly an attractive agent for PTSD since is an approved nutritional supplement with a known safety profile, thus positive results with this agent in animals could be translated into human studies. The data suggest that SAM's treatment modulates the DNA methylation profile of PTSD animals and is a candidate effective therapy to relieve two of the three characteristic pathological behaviors in PTSD in humans, exploration and social interaction.

If DNA methylation plays a causal role in PTSD, the differentially methylated genes, particularly those that exhibit correlation between levels of methylation and inter-individual differences in behavioral phenotypes are candidates for playing a role in the PTSD phenotypes susceptibility and resilience in the animals. The output of Capture bisulfite sequencing unravels candidate genes for intervention, which are likely to respond to either natural or synthetic chemicals (vitamins for example). Since the number of CG sites that were affected was large as is the case in many epigenetic studies (i.e. ⁷), whether they fall into particular functional groups was examined. It was reasoned that effective intervention should target pathways rather than particular genes that are differentially methylated. When the different upstream regulators of pathways enriched in differentially methylated genes in susceptible, resilient animals and reversed with SAM treatment were clustered, few significant networks of genes that have strong known relationship to PTSD, such as sex hormones, glucocorticoids, cytokines and others described in FIG. 6 were identified. One of the pathways that showed up as differentially altered in control, susceptible and resilient animals is the retinoic acid pathway. Interestingly RORA was previously identified in a Genome Wide Association Study (GWAS) for PTSD⁴⁷. As a proof of principle for this approach, the susceptible animals were treated with retinoic acid, a pathway revealed to be differentially methylated in susceptible animals, and the treatment was combined with SAM. Retinoic acid treatment reversed the hyperarousal behavior of PTSD animals. SAM reversed the other two behaviors, exploration and social interaction and the two treatments had synergistic effect on PTSD phenotypes. These experiments provide proof of principle for the hypothesis that DNA methylation alterations in PTSD target a wide cast of genes and therefore a combination of global modulators of either the DNA methylation machinery or pathways that are differentially methylated are a potentially new approach to understanding PTSD and treating it.

The discovery highlights the value of important networks of genes involved in disease as an effective way for revealing new therapeutics. In the example studied here, the analysis points to a combination of two approved natural products as an effective “cocktail” for treating PTSD that could be translated to treating humans

In reference to FIG. 1, “A” is a schematic description of the model. “B” is the differences in freezing behavior during the “exploration” test between control, PTSD susceptible and PTSD resilient animals. “C” is the differences in freezing behavior during the “social interaction” test between control, PTSD susceptible and PTSD resilient animals. “D” is the differences in freezing behavior during the “hyperarousal” test between control, PTSD susceptible and PTSD resilient animals. “E” is the distribution of freezing time data for exploration behavior at the second reminder time point. “F” is the distribution of freezing time data for social interaction behavior at the second reminder time point. “G” is the distribution of freezing time data for hyperarousal behavior at the second reminder time point. The dotted line is the level for freezing in the base-line value for each condition. The gray line is the mean of each subpopulation. “H” is the Pearson's correlations between exploration and social interaction (R=0.69; p<0.0001). “I” is the Pearson's correlations between exploration and hyperarousal (R=0.80; p<0.0001). “J” is the Pearson's correlations between social interaction and hyperarousal (R=0.70; p<0.0001). In reference to FIG. 2, “A” is an average DNA methylation in the three groups. “B” is the genome browser tracks showing from top to bottom sites that are differentially methylated between PTSD susceptible and control animals, between PTSD resilient and control animals, 136 sites that are commonly differentially methylated between susceptible and control animals and between resilient and control animals (hyper-methylation in red hypo-methylation in blue). Bottom lane indicates the RefSeq genes. “C” is a Venn diagram illustrating the overlap and differences in sites that are differentially methylated from controls in PTSD resilient and susceptible animals. “D” is a Heat map of clustering of the different animals by 738 sites that are differentially methylated between resilient and susceptible animals and control.

In reference to FIG. 3, “A” is the exploration test, “B” is the social interaction test, and “C” is the hyperarousal test.

In reference to FIG. 4, “A” is the genome browser tracks showing from top to bottom, sites that are differentially methylated between the PTSD susceptible animals and control, sites that are differentially methylated between SAM treated susceptible animals and susceptible animals treated with saline, 140 CG sites that are differentially methylated between PTSD susceptible animals and controls and are also affected by SAM treatment in susceptible animals. “B” is a Venn diagram showing overlap between sites that are differentially methylated between PTSD susceptible animals and controls and sites that are differentially methylated between PTSD susceptible animals and animals that were treated with SAM. 140 sites that are differentially methylated between susceptible animals and controls are reversed by SAM treatment. “C” is the genome browser tracks showing 38 sites that are differentially methylated between both PTSD susceptible animals and control (top) and between PTSD resilient and controls (second) and are reversed by SAM treatment (third). Refseq genes are shown in the bottom track. “D” is a Venn diagram showing overlap between sites that are differentially methylated from control in both susceptible and resilient animals that are reversed by SAM treatment.

In reference to FIG. 5, “A” is the correlation between differences in DNA methylation and differences in social interaction and exploration behaviors. “B” is the hierarchical clustering by DNA methylation profiles of sites that are correlated with behavior across the animals in the different treatment groups.

In reference to FIG. 6, “A” is coloring representing the p value of each group for the different “upstream-regulator” regulated pathways. Light blue represents low activation and dark blue represents high activation of the pathway. Grey represents pathways that didn't change. Yellow lines and boxes represent sex hormone dependent pathways, blue lines and boxes represent cytokine regulated pathways, pink lines and boxes represent glucocorticoids regulated pathways and green lines and boxes represent retinoic acid (vitamin A) regulated pathways. “B” is circular heat maps representing the different pathways that are influenced by a specific “upstream-upregulator” and are altered in PTSD susceptible and resistant animals. The upper circles represent the state of activity of beta-estradiol (or ESR-1 for SAM treated group) regulated genes in the susceptible group treated with saline relative to control, the resilient group treated with saline relative to control or the susceptible animals treated with SAM relative to susceptible animals treated with saline. The lowest circular heat map represents the state of activity of retinoid-related orphan receptor alpha (RORA) regulated genes in the susceptible group treated with saline only relative to control. “C” is the RORA mRNA levels in the brain Nucleus accumbens region following the third reminder (Re3) (n=4-7 in each group) showing significant differences between the susceptible groups (treated with either saline or SAM) and the other groups. Bars represent mean+SEM. “D” is the correlations between mRNA expression levels of RORA to hyperarousal freezing levels (R=0.58, ***p=0.0004).

In reference to FIG. 7, PTSD susceptible and resilient animals received two injections of SAM and retinoic acid (RA), 24 h and 1 h before the third reminder. Freezing behavior was assessed by exploration behavior in “A”, social interaction behavior in “B”, and hyperarousal behavior in “C”. The continuous grey line represents second reminder freezing levels of the susceptible group and the dotted grey line represents second reminder freezing levels of the resilient group. Bars represent freezing levels of the third reminder. A significant attenuation is observed in freezing behavior of susceptible group that was treated with SAM+RA and SAM versus susceptible group that was treated with saline in the third reminder. In the hyperarousal test only the SAM+RA was significantly attenuated (exploration: ***p<0.001, *p<0.05; social interaction: **p<0.01, *p<0.05; hyperarousal: **p<0.01). Bars represent mean+SEM; n=3-6 per group.

Although the invention has been explained in relation to its preferred embodiment, it is to be understood that many other possible modifications and variations can be made without departing from the spirit and scope of the invention.

### REFERENCES

1. Yehuda, R. Understanding Heterogeneous Effects of Trauma Exposure: Relevance to Postmortem Studies of PTSD. Psychiatry Interpers. Biol. Process. 67, 391-397 (2004).
2. Puetz, T. W., Youngstedt, S. D. & Herring, M. P. Effects of Pharmacotherapy on Combat-Related PTSD, Anxiety, and Depression: A Systematic Review and Meta-Regression Analysis. PLoS One 10, e0126529 (2015).
3. Singewald, N., Schmuckermair, C., Whittle, N., Holmes, A. & Ressler, K. J. Pharmacology of cognitive enhancers for exposure-based therapy of fear, anxiety and trauma-related disorders. Pharmacol. Ther. 149, 150-190 (2014).
4. Nestler, E. J. & Carlezon, W. A. The mesolimbic dopamine reward circuit in depression. Biol. Psychiatry 59, 1151-9 (2006).
5. Wise, R. A. Dopamine and reward: the anhedonia hypothesis 30 years on. Neurotox. Res. 14, 169-83 (2008).
6. Carlezon, W. A. & Thomas, M. J. Biological substrates of reward and aversion: a nucleus accumbens activity hypothesis. Neuropharmacology 56 Suppl 1, 122-32 (2009).
7. Admon, R. et al. Imbalanced neural responsivity to risk and reward indicates stress vulnerability in humans. Cereb. Cortex 23, 28-35 (2013).
8. Sailer, U. et al. Altered reward processing in the nucleus accumbens and mesial prefrontal cortex of patients with posttraumatic stress disorder. Neuropsychologia 46, 2836-44 (2008).
9. Elman, I. et al. Functional neuroimaging of reward circuitry responsivity to monetary gains and losses in posttraumatic stress disorder. Biol. Psychiatry 66, 1083-90 (2009).
10. Davis, M. The role of the amygdala in fear and anxiety. Annu. Rev. Neurosci. 15, 353-75 (1992).
11. Depue, R. A. & Collins, P. F. Neurobiology of the structure of personality: dopamine, facilitation of incentive motivation, and extraversion. Behav. Brain Sci. 22, 491-517; discussion 518-69 (1999).
12. Zald, D. H. The human amygdala and the emotional evaluation of sensory stimuli. Brain Res. Brain Res. Rev. 41, 88-123 (2003).
13. Hariri, A. R. The neurobiology of individual differences in complex behavioral traits. Annu. Rev. Neurosci. 32, 225-47 (2009).
14. Haber, S. N. & Knutson, B. The reward circuit: linking primate anatomy and human imaging. Neuropsychopharmacology 35, 4-26 (2010).
15. Razin, A. & Riggs, A. D. DNA methylation and gene function. Science 210, 604-10 (1980).
16. Kriaucionis, S. & Heintz, N. The nuclear DNA base 5-hydroxymethylcytosine is present in Purkinje neurons and the brain. Science 324, 929-30 (2009).
17. Jenuwein, T. & Allis, C. D. Translating the histone code. Science 293, 1074-80 (2001).
18. Day, J. J. et al. DNA methylation regulates associative reward learning. Nat. Neurosci. 16, 1445-52 (2013).
19. Levenson, J. M. et al. Evidence that DNA (cytosine-5) methyltransferase regulates synaptic plasticity in the hippocampus. J. Biol. Chem. 281, 15763-15773 (2006).
20. Miller, C. A. & Sweatt, J. D. Covalent Modification of DNA Regulates Memory Formation. Neuron 53, 857-869 (2007).
21. Feng, J. et al. Dnmt1 and Dnmt3a maintain DNA methylation and regulate synaptic function in adult forebrain neurons. Nat. Neurosci. 13, 423-30 (2010).
22. LaPlant, Q. et al. Dnmt3a regulates emotional behavior and spine plasticity in the nucleus accumbens. Nat. Neurosci. 13, 1137-43 (2010).
23. Massart, R. et al. Role of DNA Methylation in the Nucleus Accumbens in Incubation of Cocaine Craving. J. Neurosci. 35, 8042-8058 (2015).
24. Uddin, M. et al. Epigenetic and immune function profiles associated with posttraumatic stress disorder. Proc Natl Acad Sci USA 107, 9470-9475 (2010).
25. Mehta, D. et al. Childhood maltreatment is associated with distinct genomic and epigenetic profiles in posttraumatic stress disorder. Proc. Natl. Acad. Sci. U.S.A. 110, 8302-7 (2013).
26. Zannas, A. S., Provencal, N. & Binder, E. B. Epigenetics of Posttraumatic Stress Disorder: Current Evidence, Challenges, and Future Directions. Biol. Psychiatry 78, 1-9 (2015).
27. Klengel, T. et al. Allele-specific FKBP5 DNA demethylation mediates gene-childhood trauma interactions. Nat. Neurosci. 16, 33-41 (2013).
28. Chang, S. C. et al. Molecular variation at the SLC6A3 locus predicts lifetime risk of PTSD in the Detroit Neighborhood Health Study. PLoS One 7, (2012).
29. Ozer, E. J., Best, S. R., Lipsey, T. L. & Weiss, D. S. Predictors of posttraumatic stress disorder and symptoms in adults: a meta-analysis. Psychol. Bull. 129, 52-73 (2003).
30. Bonanno, G. A. Loss, trauma, and human resilience. Am. Psychol. 59, 20-28 (2004).
31. Giannoni-Pastor, A. et al. Prevalence and Predictors of Posttraumatic Stress Symptomatology Among Burn Survivors: A Systematic Review and Meta-Analysis. J. Burn Care Res. 79-89 (2016). doi:10.1097/BCR.0000000000000226
32. Kesner, Y. et al. WFS1 gene as a putative biomarker for development of post-traumatic syndrome in an animal model. Mol. Psychiatry 14, 86-94 (2009).
33. American Psychiatric Association. Diagnostic and Statistical Manual of Mental Disorders, 5th Edition (DSM-5). Diagnostic Stat. Man. Ment. Disord. 4th Ed. TR. 280 (2013). doi:10.1176/appi.books.9780890425596.744053
34. McDermott, C. M., Liu, D., Ade, C. & Schrader, L. A. Estradiol replacement enhances fear memory formation, impairs extinction and reduces COMT expression levels in the hippocampus of ovariectomized female mice. Neurobiol. Learn. Mem. 118, 167-177 (2015).
35. Domschke, K. Patho-genetics of posttraumatic stress disorder. Psychiatr Danub 24, 267-273 (2012).
36. Digangi, J., Guffanti, G., McLaughlin, K. A. & Koenen, K. C. Considering trauma exposure in the context of genetics studies of posttraumatic stress disorder: a systematic review. Biol. Mood Anxiety Disord. 3, 2 (2013).
37. Binder, E. B. et al. Polymorphisms in FKBP5 are associated with increased recurrence of depressive episodes and rapid response to antidepressant treatment. Nat. Genet. 36, 1319-1325 (2004).
38. Alim, T. N. et al. Trauma, resilience, and recovery in a high-risk African-American population. Am. J. Psychiatry 165, 1566-75 (2008).
39. Collishaw, S. et al. Resilience to adult psychopathology following childhood maltreatment: Evidence from a community sample. Child Abuse Negl. 31, 211-229 (2007).
40. Luthar, S. S., Cicchetti, D. & Becker, B. The Construct of Resilience: A Critical Evaluation and Guidelines for Future Work. Child Dev. 71, 543-562 (2000).
41. Wingo, A. P., Ressler, K. J. & Bradley, B. Resilience characteristics mitigate tendency for harmful alcohol and illicit drug use in adults with a history of childhood abuse: a cross-sectional study of 2024 inner-city men and women. J. Psychiatr. Res. 51, 93-9 (2014).
42. Kessler, R. C., Sonnega, A., Bromet, E., Hughes, M. & Nelson, C. B. Posttraumatic stress disorder in the National Comorbidity Survey. Arch. Gen. Psychiatry 52, 1048-60 (1995).
43. Bonanno, G. A. Loss, trauma, and human resilience: have we underestimated the human capacity to thrive after extremely aversive events? Am. Psychol. 59, 20-8 (2004).
44. Moore, S. A. Cognitive abnormalities in posttraumatic stress disorder. Curr. Opin. Psychiatry 22, 19-24 (2009).
45. Yehuda, R. & Bierer, L. M. The relevance of epigenetics to PTSD: implications for the DSM-V. J. Trauma. Stress 22, 427-34 (2009).
46. Alvarado, S., Rajakumar, R., Abouheif, E. & Szyf, M. Epigenetic variation in the Egfr gene generates quantitative variation in a complex trait in ants. Nat. Commun. 6, 1-9 (2015).
47. Logue, M. W. et al. A genome-wide association study of post-traumatic stress disorder identifies the retinoid-related orphan receptor alpha (RORA) gene as a significant risk locus. Mol. Psychiatry 18, 937-942 (2012).
48. Akalin, A., et al. methylKit: a comprehensive R package for the analysis of genome-wide DNA methylation profiles. Genome Biol 13, R87 (2012).
49. Razin, A. & Riggs, A. D. DNA methylation and gene function. Science 210, 604-610 (1980).
50. Lister, R., et al. Global epigenomic reconfiguration during mammalian brain development. Science 341, 1237905 (2013).
51. Szyf, M. The genome- and system-wide response of DNA methylation to early life adversity and its implication on mental health. Can J Psychiatry 58, 697-704 (2013).
52. Klengel, T., et al. Allele-specific FKBP5 DNA demethylation mediates gene-childhood trauma interactions. Nat Neurosci 16, 33-41 (2013).
53. Chik, F., Machnes, Z. & Szyf, M. Synergistic anti breast cancer effect of a combined treatment with the methyl donor S-adenosyl methionine (SAM) with the DNA methylation inhibitor 5-aza-2′-deoxycytidine. Carcinogenesis (2013).
54. Massart, R., et al. Role of DNA methylation in the nucleus accumbens in incubation of cocaine craving. J Neurosci 35, 8042-8058 (2015).

What is claimed is:

1. A DNA methylation signature of post-traumatic stress disorder (PTSD) in brains of susceptible, resilient animals methylated in response to trauma, and S-adensoyl methionine (SAM) treated animals for deriving targets for PTSD therapeutics.

2. A pathway analysis of said DNA methylation landscape to derive novel targets for therapeutic interventions such as retinoic acid pathway or estrogen receptor pathways.

3. A method of treatment of PTSD comprised of Epigenetic modulators using general DNA methylation modulators such as SAM.

4. A method of treatment of PTSD comprised of retinoic acid or vitamin A and its natural and synthetic analogs such as all-trans-retinoic acid (Tretinoin), 9-cis-retinoic acid (Alitretinoin), and 13-cis-retinoic acid (Isotretinoin) to treat PTSD.

5. A method of treatment of PTSD comprised of a combination of S-adenosylmethionine and retinoic acid or vitamin A and its synthetic and natural analogs such as Tretinoin, Alitretinoin, and Isotretinoin.

6. A method of treatment of PTSD comprised of a combination of behavioral therapy and S-adenosylmethionine and retinoic acid.
